(19) United States
(12) Patent Application Publication
(10) Pub. No.: US 2025/0118444 A1
(43) Pub. Date: Apr. 10, 2025

(54) SYSTEMS AND METHODS OF GENERATING MEDICAL CONCORDANCE SCORES

(71) Applicant: PROSCIA INC., Philadelphia, PA (US)

(72) Inventors: Julianna IANNI, Merchantville, NJ (US); Vaughn SPURRIER, Philadelphia, PA (US); Sean GRULLON, Philadelphia, PA (US)

(21) Appl. No.: 18/835,238

(22) PCT Filed: Feb. 3, 2023
(86) PCT No.: PCT/US2023/012280

§ 371 Date: Aug. 1, 2024

Related U.S. Application Data
(60) Provisional application No. 63/306,714, filed on Feb. 4, 2022.

(51) Int. Cl. G16H 50/30; G16H 10/60; G16H 15/00

(52) U.S. Cl. CPC G16H 50/30; G16H 10/60; G16H 15/00

(57) ABSTRACT

Examples may provide an electronic neural network that has been trained on a set of training data that comprises a plurality of reference subject medical data sets that are each labeled with a medical determination and are each assigned a ground truth concordance score generated by a plurality of experts in which a value of a given ground truth concordance score comprises a fraction of the plurality of experts, if any, that are in accord with the medical determination label of a given reference subject medical data set in the plurality of reference subject medical data sets. The electronic neural network is configured to provide an output concordance score of the medical determination being indicated by a test subject medical data set.

### CROSS-REFERENCE TO RELATED APPLICATIONS

This application is a national stage application under 35 U.S.C. § 371 of PCT/US2023/012280, filed Feb. 3, 2023, and entitled “Systems and Methods of Generating Medical Concordance Scores,” which claims the benefit of U.S. Provisional Patent Application Ser. No. 63/306,714, filed Feb. 4, 2022, the disclosures of which are incorporated herein by reference.

### FIELD

This disclosure relates generally to machine learning, e.g., in the context of medical applications, such as pathology.

### BACKGROUND

In medical practice generally, and more particularly in pathology, experts often agree on a given diagnosis. In some cases, however, the evidence supporting a possible diagnosis, clinical finding, or clinical recommendation is unclear. In some of these instances, for example, the presenting case is simply more complex than others. In such cases, it is not uncommon for medical experts to disagree on the diagnosis. This disagreement is important when such information is available because it highlights cases where greater scrutiny is likely needed to arrive at an accurate diagnosis. In many instances, however, patients receive diagnoses only from a single expert. This tends to increase the probability of misdiagnosis and consequent administration of inappropriate therapies, particularly in uncertain or otherwise complex cases. Accordingly, in these instances, the decision-making of the single expert would be further informed by knowledge of a likely concordance or discordance level among a group of multiple experts in a given case under consideration.

In medical and scientific literature, the rate of expert agreement is often referred to as inter-rater agreement, interobserver agreement, or the rate of concordance or its opposite, discordance. When all experts agree, or a multi-expert opinion or diagnosis has been reached by other means (e.g., a majority-vote, or review and discussion by a board of experts), this is referred to as consensus. As noted above, having an estimate of the degree of expert concordance or consensus would be valuable. In the practice of pathology, for example, the most challenging cases are often sent to tumor review boards to be decided on by a panel of experts. In situations where such a panel is not available, a case may be sent for additional review by a second expert before a diagnosis is rendered. Moreover, in many areas, laboratories are required to perform quality reviews in which some subset of cases is selected to undergo re-review to confirm that accurate diagnoses were rendered. Particularly, in cases that do not receive any of these double-reviews, it would be beneficial to consult a panel of experts. This would flag cases that were originally misdiagnosed, or cases that merit further examination or testing to confirm accurate diagnosis. Currently, if a pathologist or other healthcare provider knows that he or she is dealing with a difficult case, they may order additional tests or stains on that case to increase confidence in their diagnosis and decrease the possibility of misdiagnosis. An ability to consult a panel of experts would also be of benefit in these cases.

Accordingly, it is apparent that there is a need for additional methods of providing concordance information to healthcare providers to further inform diagnostic, prognostic, and therapeutic decision-making.

### SUMMARY

The present disclosure provides, in certain aspects, an artificial intelligence (AI) system capable of predicting the results of consulting a panel of experts and delivering a decision or a score based on the degree to which the experts would agree on a particular diagnosis or opinion. These and other aspects will be apparent upon a complete review of the present disclosure, including the accompanying figures.

According to various embodiments, a computer-implemented method of generating a medical concordance score from a test subject medical data set is presented. The method includes: passing the test subject medical data set through an electronic neural network, wherein the electronic neural network has been trained on a set of training data that comprises a plurality of reference subject medical data sets that are each labeled with a medical determination and are each assigned a ground truth concordance score generated by a plurality of experts, wherein a value of a given ground truth concordance score comprises a fraction of the plurality of experts, if any, that are in accord with the medical determination label of a given reference subject medical data set in the plurality of reference subject medical data sets; and outputting from the electronic neural network a concordance score of the medical determination being indicated by the test subject medical data.

Various optional features of the above embodiments include the following. The plurality of experts comprises human experts, machine experts, or a combination of human and machine experts. The plurality of experts comprises at least about three, at least about four, at least about five, at least about six, at least about seven, at least about eight, at least about nine, at least about ten, or more experts. Labeling the test subject medical data set with the medical determination prior to or when passing the test subject medical data set through the electronic neural network. The medical determination comprises a diagnosis of a disease, condition, or disorder. The medical determination comprises a prognosis of a disease, condition, or disorder. The medical determination comprises a recommended treatment plan for a diagnosed disease, condition, or disorder. The medical determination comprises an atypical status, a benign status, or a malignant status. The medical determination comprises an in situ melanoma status or an invasive melanoma status. The medical determination comprises a Gleason score. The medical determination comprises a survival quantification. The medical determination comprises a therapy response. The medical determination comprises a dermatopathological class, prognosis, or diagnosis. Ordering one or more medical tests for, and/or administering one or more therapies to, the test subject when the concordance score of the medical determination being indicated by the test subject medical data set varies from a predetermined threshold value. The one or more medical tests comprise at least one histological stain of a sample obtained from the test subject. Discontinuing administering one or more therapies to the test subject when the concordance score of the medical determination being indicated by the test subject medical data set varies from a predetermined threshold value. Prioritizing the test subject's case among other cases based on the concordance score being indicated by the test subject medical data. Flagging, alerting, and/or prioritizing for review cases, which have concordance scores that conflict with the rendered diagnosis, prognosis, or the like in a medical or laboratory information system. Generating or updating at least a portion of a medical report for the test subject when the concordance score of the medical determination being indicated by the test subject medical data set varies from a predetermined threshold value. The test and reference subject medical data sets comprise images of histopathology slides. The images comprise whole slide images (WSI). The test and reference subject medical data sets comprise images selected from the group consisting of: a magnetic resonance (MR) image, a computed tomography (CT) image, a single photon emission computed tomography (SPECT) image, a positron emission tomography (PET) image, and a microscopy image. Removing at least one extraneous property in the test and reference subject medical data sets. The electronic neural network comprises at least one layer that performs a regression operation to generate the medical concordance score. The electronic neural network has been trained using a multiple instance learning (MIL) model.

According to various embodiments, a system for generating a medical concordance score from a test subject medical data set using an electronic neural network is presented. The system includes a processor; and a memory communicatively coupled to the processor, the memory storing instructions which, when executed on the processor, perform operations including: passing the test subject medical data set through an electronic neural network, wherein the electronic neural network has been trained on a set of training data that comprises a plurality of reference subject medical data sets that are each labeled with a medical determination and are each assigned a ground truth concordance score generated by a plurality of experts, wherein a value of a given ground truth concordance score comprises a fraction of the plurality of experts, if any, that are in accord with the medical determination label of a given reference subject medical data set in the plurality of reference subject medical data sets; and outputting from the electronic neural network a concordance score of the medical determination being indicated by the test subject medical data set.

Various optional features of the above embodiments include the following. The plurality of experts comprises human experts, machine experts, or a combination of human and machine experts. The plurality of experts comprises at least about three, at least about four, at least about five, at least about six, at least about seven, at least about eight, at least about nine, at least about ten, or more experts. The test subject medical data set is labeled with the medical determination prior to or when passing the test subject medical data set through the electronic neural network. The medical determination comprises a diagnosis of a disease, condition, or disorder. The medical determination comprises a prognosis of a disease, condition, or disorder. The medical determination comprises a recommended treatment plan for a diagnosed disease, condition, or disorder. The medical determination comprises an atypical status, a benign status, or a malignant status. The medical determination comprises an in situ melanoma status or an invasive melanoma status. The medical determination comprises a Gleason score. The medical determination comprises a survival quantification. The medical determination comprises a therapy response. The medical determination comprises a dermatopathological class, prognosis, or diagnosis. The system orders one or more medical tests for, and/or recommends administering one or more therapies to, the test subject when the concordance score of the medical determination being indicated by the test subject medical data set varies from a predetermined threshold value. The one or more medical tests comprise at least one histological stain of a sample obtained from the test subject. The system recommends discontinuing administering one or more therapies to the test subject when the concordance score of the medical determination being indicated by the test subject medical data set varies from a predetermined threshold value. The system generates or updates at least a portion of a medical report for the test subject when the concordance score of the medical determination being indicated by the test subject medical data set varies from a predetermined threshold value. The test and reference subject medical data sets comprise images of histopathology slides. The images comprise whole slide images (WSI). The test and reference subject medical data sets comprise images selected from the group consisting of: a magnetic resonance (MR) image, a computed tomography (CT) image, a single photon emission computed tomography (SPECT) image, a positron emission tomography (PET) image, and a microscopy image. The system removes at least one extraneous property in the test and reference subject medical data sets. The electronic neural network comprises at least one layer that performs a regression operation to generate the medical concordance score. The electronic neural network has been trained using a multiple instance learning (MIL) model.

### DRAWINGS

The above and/or other aspects and advantages will become more apparent and more readily appreciated from the following detailed description of examples, taken in conjunction with the accompanying drawings, in which:

FIG. 1 is a flow chart that schematically shows exemplary method steps of generating a medical concordance score from a test subject medical data set according to some aspects disclosed herein; and

FIG. 2 is a schematic diagram of an exemplary system suitable for use with certain aspects disclosed herein.

### DEFINITIONS

In order for the present disclosure to be more readily understood, certain terms are first defined below. Additional definitions for the following terms and other terms may be set forth throughout the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

It is also to be understood that the terminology used herein is for the purpose of describing particular embodiments only and is not intended to be limiting. Further, unless defined otherwise, all technical and scientific terms used herein have the same meaning as commonly understood by one of ordinary skill in the art to which this disclosure pertains. In describing and claiming the methods, systems, and computer readable media, the following terminology, and grammatical variants thereof, will be used in accordance with the definitions set forth below.

Classifier. As used herein, “classifier” generally refers to algorithm computer code that receives, as input, test data and produces, as output, a classification of the input data as belonging to one or another class.

Concordance score: As used herein, “concordance score” in the context of medical data refers to a value or measure that represents a degree, level, fraction, or proportion of consensus or agreement (or lack thereof (i.e., discordance)) regarding a medical determination related to a subject among a plurality of experts.

Data set: As used herein, “data set” refers to a group or collection of information, values, or data points related to or associated with one or more objects, records, and/or variables. In some embodiments, a given data set is organized as, or included as part of, a matrix or tabular data structure. In some embodiments, a data set is encoded as a feature vector corresponding to a given object, record, and/or variable, such as a given test or reference subject. For example, a medical data set for a given subject can include one or more observed values of one or more variables associated with that subject.

Electronic neural network: As used herein, “electronic neural network” refers to a machine learning algorithm or model that includes layers of at least partially interconnected artificial neurons (e.g., perceptrons or nodes) organized as input and output layers with one or more intervening hidden layers that together form a network that is or can be trained to classify data, such as test subject medical data sets (e.g., medical images or the like).

Expert: As used herein, “expert” refers to an entity that is trained to at least to a selected threshold level regarding a given knowledge domain. In some embodiments, an expert is a “human expert” such as a healthcare provides (e.g., a pathologist, radiologist, oncologist, or the like). In some embodiments, an expert is a “machine expert” such as a machine learning model that has been trained as to one or more aspects of the given knowledge domain.

Labeled: As used herein, “labeled” in the context of data sets or points refers to data that is classified as, or otherwise associated with, having or lacking a given characteristic or property.

Machine Learning Algorithm: As used herein, “machine learning algorithm” generally refers to an algorithm, executed by computer, that automates analytical model building, e.g., for clustering, classification or pattern recognition. Machine learning algorithms may be supervised or unsupervised. Learning algorithms include, for example, artificial neural networks (e.g., back propagation networks), discriminant analyses (e.g., Bayesian classifier or Fisher's analysis), multiple-instance learning (MIL), support vector machines, decision trees (e.g., recursive partitioning processes such as CART-classification and regression trees, or random forests), linear classifiers (e.g., multiple linear regression (MLR), partial least squares (PLS) regression, and principal components regression), hierarchical clustering, and cluster analysis. A dataset on which a machine learning algorithm learns can be referred to as “training data.” A model produced using a machine learning algorithm is generally referred to herein as a “machine learning model.”

Medical determination: As used herein, “medical determination” refers to a conclusion as to the presence or absence of a given disease, condition, or disorder and/or a prognosis related to that conclusion.

Multiple instance learning: As used herein, “multiple instance learning” or “MIL” refers to a type of supervised machine learning in which the algorithm is trained with a set of labeled bags or groups of data points in which individual instances or observations in those bags are unlabeled, and then classifies individual, or bags of, test or unknown instances with a label.

Subject. As used herein, “subject” or “test subject” refers to an animal, such as a mammalian species (e.g., human) or avian (e.g., bird) species. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or pathology or a predisposition to the disease or pathology, or an individual that is in need of therapy or suspected of needing therapy. The terms “individual” or “patient” are intended to be interchangeable with “subject.” A “reference subject” refers to a subject known to have or lack specific properties (e.g., a known pathology, such as melanoma and/or the like).

Value: As used herein, “value” generally refers to an entry in a dataset that can be anything that characterizes the feature to which the value refers. This includes, without limitation, numbers, words or phrases, symbols (e.g., + or −) or degrees.

### Description of the Embodiments

Reference will now be made in detail to example implementations. These embodiments are described in sufficient detail to enable those skilled in the art to practice the invention and it is to be understood that other embodiments may be utilized and that changes may be made without departing from the scope of the invention. The following description is, therefore, merely exemplary.

### I. Introduction

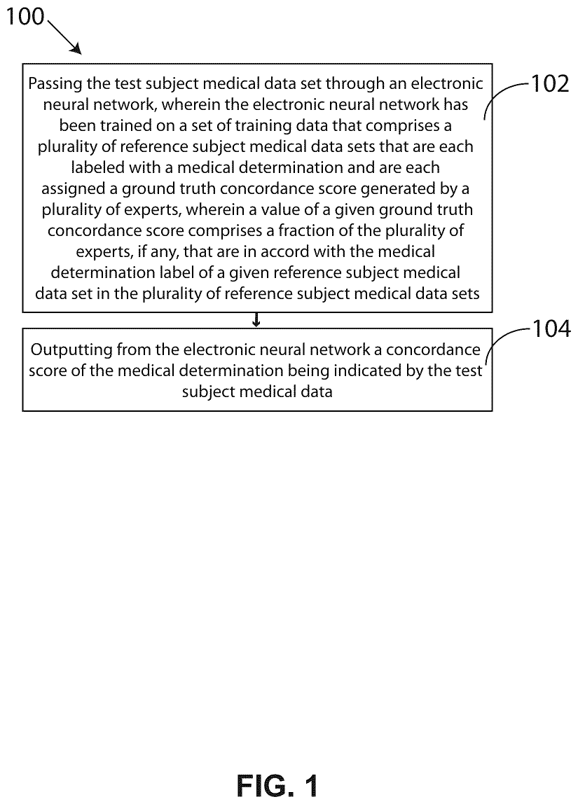

In some aspects, the present disclosure provides computer-implemented methods of generating a medical concordance score from a test subject medical data set. To illustrate, FIG. 1 is a flow chart that schematically shows certain of these exemplary method steps. As shown, method 100 includes passing a test subject medical data set through an electronic neural network (step 102). The electronic neural network has been trained on a set of training data that comprises a plurality of reference subject medical data sets that are each labeled with a medical determination and are each assigned a ground truth concordance score generated by a plurality of experts (e.g., human and/or machine experts). A value of a given ground truth concordance score typically comprises a fraction of the plurality of experts, if any, that are in accord with the medical determination label of a given reference subject medical data set in the plurality of reference subject medical data sets. In some of these embodiments, for example, a given ground truth concordance score is based on or correlated with that fraction of the plurality of experts but is not necessarily equal to that score. In some embodiments, the electronic neural network comprises at least one layer that performs a regression operation to generate the medical concordance score. In some embodiments, the electronic neural network has been trained using a multiple instance learning (MIL) model. Method 100 also includes outputting from the electronic neural network a concordance score of the medical determination being indicated by the test subject medical data.

Essentially any medical determination is optionally adapted for use with the methods and other aspects disclosed herein. In some embodiments, for example, the medical determination comprises a diagnosis and/or a prognosis of a disease, condition, or disorder. In some embodiments, the medical determination comprises a recommended treatment plan for a diagnosed disease, condition, or disorder. In some embodiments, the medical determination comprises an atypical status, a benign status, or a malignant status. In some embodiments, the medical determination comprises an in situ melanoma status or an invasive melanoma status. In some embodiments, the medical determination comprises a Gleason score. In some embodiments, the medical determination comprises a survival quantification. In some embodiments, the medical determination comprises a therapy response. In some embodiments, the medical determination comprises a dermatopathological class, prognosis, or diagnosis.

In some embodiments, method 100 further includes ordering medical tests for, and/or administering therapies to, the test subject when the concordance score of the medical determination being indicated by the test subject medical data set varies from a predetermined threshold value. In some of these embodiments, for example, the medical tests comprise a histological stain of a sample obtained from the test subject. In some embodiments, method 100 further includes discontinuing administering one or more therapies to the test subject when the concordance score of the medical determination being indicated by the test subject medical data set varies from a predetermined threshold value. In some embodiments, method 100 further includes generating or updating at least a portion of a medical report for the test subject when the concordance score of the medical determination being indicated by the test subject medical data set varies from a predetermined threshold value.

In some embodiments, the test and reference subject medical data sets comprise images of histopathology slides. The images comprise whole slide images (WSI). The test and reference subject medical data sets comprise images, such as magnetic resonance (MR) images, computed tomography (CT) images, single photon emission computed tomography (SPECT) images, positron emission tomography (PET) images, and microscopy images, among other image types. In some embodiments, method 100 includes removing extraneous properties in, or otherwise preprocessing, the test and reference subject medical data sets.

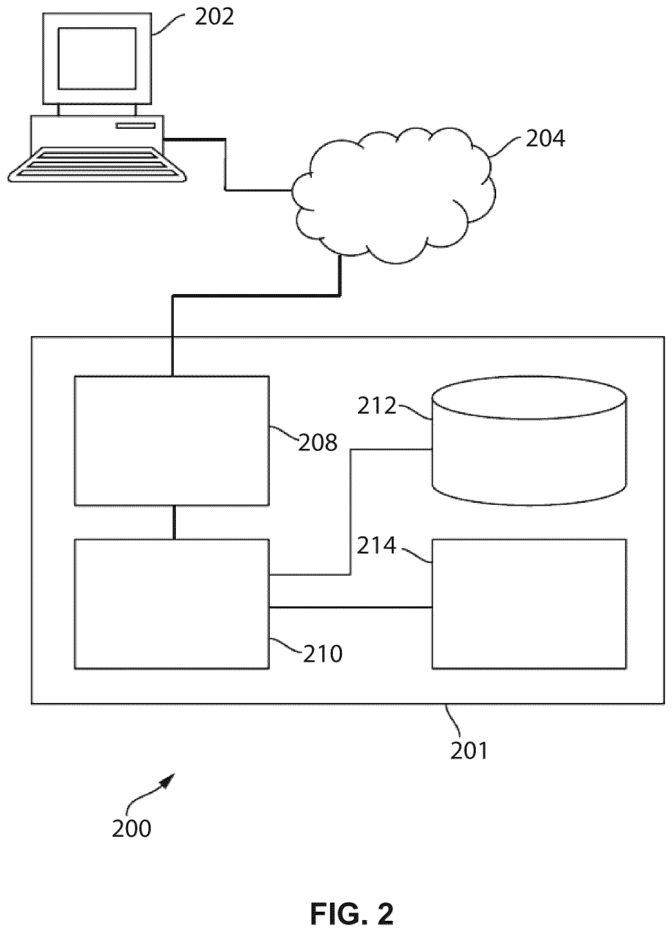

FIG. 2 is a schematic diagram of a hardware computer system 200 suitable for implementing various embodiments. For example, FIG. 2 illustrates various hardware, software, and other resources that can be used in implementations of any of methods disclosed herein, including method 100 and/or one or more instances of an electronic neural network. System 200 includes training corpus source 202 and computer 201. Training corpus source 202 and computer 201 may be communicatively coupled by way of one or more networks 204, e.g., the internet.

Training corpus source 202 may include an electronic clinical records system, such as an LIS, a database, a compendium of clinical data, or any other source of supra-images suitable for use as a training corpus as disclosed herein. As used herein, the term “supra-image” embraces any type of specimen in any field, not limited to pathology, where the problem at hand involves labels for groups of components, e.g., a set of satellite images or a set of 2D radiology images that may represent a large 3D volume. Each supra-image is composed of one or more “images”, which may be whole-slide images, e.g., representing a biopsy. Due to hardware volatile memory storage limitations, each constituent image of a supra-image may be broken down into a number of tiles, which may be, e.g., 128 pixels by 128 pixels. Such tiles are examples of “components” as that term is used herein. According to some embodiments, each component is implemented as a vector, such as a feature vector, that represents a respective tile. Thus, the term “component” refers to both a tile and a feature vector representing a tile.

Computer 201 may be implemented as any of a desktop computer, a laptop computer, can be incorporated in one or more servers, clusters, or other computers or hardware resources, or can be implemented using cloud-based resources. Computer 201 includes volatile memory 214 and persistent memory 212, the latter of which can store computer-readable instructions, that, when executed by electronic processor 210, configure computer 201 to perform any of the methods disclosed herein, including method 100, and/or form or store any electronic neural network, and/or perform any classification technique as described herein. Computer 201 further includes network interface 208, which communicatively couples computer 201 to training corpus source 202 via network 204. Other configurations of system 200, associated network connections, and other hardware, software, and service resources are possible.

Certain embodiments can be performed using a computer program or set of programs. The computer programs can exist in a variety of forms both active and inactive. For example, the computer programs can exist as software program(s) comprised of program instructions in source code, object code, executable code or other formats; firmware program(s), or hardware description language (HDL) files. Any of the above can be embodied on a transitory or non-transitory computer readable medium, which include storage devices and signals, in compressed or uncompressed form. Exemplary computer readable storage devices include conventional computer system RAM (random access memory), ROM (read-only memory), EPROM (erasable, programmable ROM), EEPROM (electrically erasable, programmable ROM), and magnetic or optical disks or tapes.

### II. Description of Example Embodiments

Some embodiments provide methods for delivering a decision or score (“concordance score”) for the distinction between benign and malignant melanocytic lesions (i.e., benign lesions vs. melanoma). However, applications of the methods disclosed herein are not limited to melanoma diagnosis and instead can be implemented to address other medical determinations. More specifically, the concordance scoring approaches disclosed herein can be applied to other pathologies, such as colon lesions or breast lesions. Furthermore, although some embodiments of these approaches are developed with digitized H&E-stained histopathology slides and are based on multiple instance learning (MIL), such concordance approaches can be applied in domains outside of digital pathology where deriving a consensus opinion or the opinion of a panel of experts (humans), or of a panel of (non-human) evidence-points (e.g. sensors) is important, such as:
Concordance Scoring in large radiology images, such as MRIs or other medical images.
Concordance Scoring of High-content microscopy images for drug activity.
Combining different sensor types for landmine detection through ground-penetrating radar. See, e.g., chapter 7 of Khalifa, A. B. (2015). MULTIPLE INSTANCE FUZZY INFERENCE, Doctoral Dissertation, University of Louisville, which is incorporated by reference in its entirety, for a discussion of how different sensor types do not agree on what constitutes a landmine.
Scoring predictions based on images from multiple satellites—e.g. detecting the presence of a water source in a given image or location.

In some embodiments, the H&E melanoma concordance scoring application is an algorithm designed to generate a score that correlates with increasing dermatopathologist concordance on a diagnosis of malignancy. The maximum value correlates with near-certainty of complete consensus among dermatopathologists that a melanocytic lesion is malignant. The minimum value correlates with near-certainty of complete consensus among dermatopathologists that a melanocytic lesion is benign. With increasing score, there is an increased likelihood of higher concordance that a specimen is malignant. The algorithm runs on whole slide images generated from formalin-fixed paraffin-embedded (FFPE) hematoxylin and eosin (H&E) stained tissue. The system is intended for use as an adjunct to the pathologist in the distinction between benign and malignant melanocytic specimens, but can also be used to flag cases that have been misdiagnosed for re-review.

The melanoma concordance score estimates dermatopathologist concordance and is interpretable as dermatopathologist concordance, in other words, what percentage of dermatopathologists agree on the presence of malignant melanoma. This is particularly important for melanoma, as there is a high rate of disagreement of invasive melanoma diagnoses (as high as 40%, see Elmore et al. Pathologists' diagnosis of invasive melanoma and melanocytic proliferations: observer accuracy and reproducibility study. BMJ, 357, 2017, which is incorporated by reference.)

### Data and Ground Truth

The melanoma concordance scoring AI system is trained with H&E stained whole slide images corresponding to specimens. These melanocytic lesions are reviewed by multiple dermatopathologists, who have rendered a diagnostic opinion on each specimen. The ground-truth concordance score is calculated to be the fraction of dermatopathologists who agree that the primary diagnosis is any variant of melanoma in situ or invasive melanoma. A value of 1.0 means complete agreement from dermatopathologists on melanoma in situ/invasive melanoma, and a value of 0.0 means complete agreement that the specimen is benign.

### Preprocessing & Embedding

Each specimen was first segmented into tissue-containing regions, and subdivided into 128×128 pixel tiles, extracted at an objective power of 10×. Each tile was passed through quality control and embedding components. Optionally, the system can use different size tiles, or no tile images at all, and main objective power used can be different.

Quality control consisted of ink filtering and blur filtering. Pen ink is common in labs migrating their workload from glass slides to WSIs where the location of possible malignancy was marked. This pen ink represented a biased distractor signal in training the system that is highly correlated with malignant or high-risk pathologies. Tiles containing pen ink were identified by a weakly supervised model trained to detect inked slides. These tiles were removed from the training and validation data and before inference on the test set. Areas of the image that were out of focus due to scanning errors were also removed by setting a threshold on the variance of the Laplacian over each tile. In some embodiments, the system does not include quality control, or includes different types of quality control or adapts different methods of performing the quality control.

In order to capture higher-level features in the WSI tiles, an embedder was trained in the SimCLR framework (Chen, Ting, et al. “A simple framework for contrastive learning of visual representations.” International conference on machine learning. PMLR, 2020). The tiles were propagated through the embedder trained with a ResNet50 backbone (He, Kaiming, et al. “Deep residual learning for image recognition.” Proceedings of the IEEE conference on computer vision and pattern recognition. 2016) to embed each input tile into 2048 channel vectors. Optionally, the system can operate with a different (or multiple) feature embedders, or no embedder at all.

### Model Architecture

The melanoma scoring architecture consists of four fully-connected layers (two layers of 1024 channels each, followed by two of 512 channels each). Each neuron in the three layers after the input layer was ReLU activated. In some embodiments, other activation functions are utilized, such as sigmoid or tanh functions. There is one final fully connected layer that performs a linear regression and provides a concordance prediction in the range 0-1. If the direct model output is not in the range 0-1 or a different range is desired, this score can be modified in post-processing. In some embodiments, other model architectures are utilized.

### Objective Function

Concordance scoring is a regression task rather than a classification task. As such, a number of regression objective functions are supported, but a default objective function is the Root Mean Squared Error Loss function:

$$R M S E = \sqrt{\frac{{\sum }_{i = 1}^{N} {‖ y ( i ) − \hat{y} ( i ) ‖}^{2}}{N}} ,$$

The system can also support, for example, the Mean Absolute Error (MAE):

$$M A E = \frac{1}{n} {\sum }_{j = 1}^{n} \vert {y}_{j} - {\hat{y}}_{j} \vert$$

### Multiple Instance Learning and Model Training

As WSIs are very high in resolution and will not fit into memory of current systems, the models are trained in a multiple instance learning (MIL) paradigm. MIL, which uses weak supervision, helps with very large images (or other types of data). Here each embedded tile was treated as an instance of a bag containing all quality-assured tiles of a specimen. A model architecture and attention mechanism similar to that used by Ilse and Welling (Ilse et al., “Attention-based Deep Multiple Instance Learning.” 2018. arXiv: 1802.04712v4), can be employed, but adapted such that the final layer performs linear regression instead of a classification. Instead of a class label, define the ground truth label of the bag or specimen as the concordance rate of dermatopathologists who reviewed the specimen (a continuous number).

Embedded tiles were aggregated using sigmoid-activated attention heads (Lu, Ming Y., et al. “Data-efficient and weakly supervised computational pathology on whole-slide images.” Nature Biomedical Engineering 5.6 (2021): 555-570). A different function for attention is optionally utilized. To help prevent overfitting, the training dataset consisted of augmented versions of the tiles. Augmentations were generated with the following augmentation strategies: random variations in brightness, hue, contrast, saturation, (up to a maximum of 15%), Gaussian noise with 0.001 variance, and random 90 degree image rotations. Optionally, a different augmentation strategy, or no augmentation, is utilized.

### Outputs, Use and Display

The AI system directly outputs a score. This score can be delivered directly to the user, or thresholded and converted into a decision (e.g. benign or malignant). A similar system can output multiple scores-perhaps in tandem predicting both diagnostic agreement and prognostic agreement. This score can be displayed to a user in an image management system, and used to aid in diagnosis. It can also be used to sort and prioritize which specimens or cases need review first, based on likelihood of malignancy. Another use can be to allow flagging and subsequent review of cases in which a diagnosis input to the image management system or a lab information system did not match with the predicted degree of concordance that a specimen was malignant.

In some implementations, a similar system can output regions of interest (heatmaps or other annotations) associated with malignancy or associated with the model's prediction of malignancy or concordance score. The system can output a score for a single slide, for an entire specimen, or for an entire case.

The score can also be used in conjunction with the image management system to perform automatic case assignment (e.g., determine which pathologist should review a case).

In some embodiments, the score or scores can additionally be used to suggest, recommend, or automatically trigger additional testing or other action, for example the ordering of additional stains or a genetic test. It can also serve as a companion diagnostic, the results of which are required in order to recommend a particular therapy or treatment decision and which can be tied to a specific therapeutic compound.

As an additional option, the score can be incorporated as a precursor (“reflex test”) or as a sub-component of another test. For example, the results of this AI system feed into a combined-test that incorporates the results of a multigene assay (e.g., Castle MelanomaDx or the like)—also a score—to enhance the performance of the test or deliver additional information.

Some further aspects are defined in the following clauses:
Clause 1: A computer-implemented method of generating a medical concordance score from a test subject medical data set, the method comprising: passing the test subject medical data set through an electronic neural network, wherein the electronic neural network has been trained on a set of training data that comprises a plurality of reference subject medical data sets that are each labeled with a medical determination and are each assigned a ground truth concordance score generated by a plurality of experts, wherein a value of a given ground truth concordance score comprises a fraction of the plurality of experts, if any, that are in accord with the medical determination label of a given reference subject medical data set in the plurality of reference subject medical data sets; and, outputting from the electronic neural network a concordance score of the medical determination being indicated by the test subject medical data, thereby generating the medical concordance score from the test subject medical data set.
Clause 2: The method of Clause 1, wherein the plurality of experts comprises human experts, machine experts, or a combination of human and machine experts.
Clause 3: The method of Clause 1 or Clause 2, wherein the plurality of experts comprises at least about three, at least about four, at least about five, at least about six, at least about seven, at least about eight, at least about nine, at least about ten, or more experts.
Clause 4: The method of any of Clauses 1-3, comprising labeling the test subject medical data set with the medical determination prior to or when passing the test subject medical data set through the electronic neural network.
Clause 5: The method of any of Clauses 1-4, wherein the medical determination comprises a diagnosis of a disease, condition, or disorder.
Clause 6: The method of any of Clauses 1-5, wherein the medical determination comprises a prognosis of a disease, condition, or disorder.
Clause 7: The method of any of Clauses 1-6, wherein the medical determination comprises a recommended treatment plan for a diagnosed disease, condition, or disorder.
Clause 8: The method of any of Clauses 1-7, wherein the medical determination comprises an atypical status, a benign status, or a malignant status.
Clause 9: The method of any of Clauses 1-8, wherein the medical determination comprises an in situ melanoma status or an invasive melanoma status.
Clause 10: The method of any of Clauses 1-9, wherein the medical determination comprises a Gleason score.
Clause 11: The method of any of Clauses 1-10, wherein the medical determination comprises a survival quantification.
Clause 12: The method of any of Clauses 1-11, wherein the medical determination comprises a therapy response.
Clause 13: The method of any of Clauses 1-12, wherein the medical determination comprises a dermatopathological class, prognosis, or diagnosis.
Clause 14: The method of any of Clauses 1-13, further comprising ordering one or more medical tests for, and/or administering one or more therapies to, the test subject when the concordance score of the medical determination being indicated by the test subject medical data set varies from a predetermined threshold value.
Clause 15: The method of any of Clauses 1-14, wherein the one or more medical tests comprise at least one histological stain of a sample obtained from the test subject.
Clause 16: The method of any of Clauses 1-15, further comprising discontinuing administering one or more therapies to the test subject when the concordance score of the medical determination being indicated by the test subject medical data set varies from a predetermined threshold value.
Clause 17: The method of any of Clauses 1-16, further comprising generating or updating at least a portion of a medical report for the test subject when the concordance score of the medical determination being indicated by the test subject medical data set varies from a predetermined threshold value.
Clause 18: The method of any of Clauses 1-17, wherein the test and reference subject medical data sets comprise images of histopathology slides.
Clause 19: The method of any of Clauses 1-18, wherein the images comprise whole slide images (WSI).
Clause 20: The method of any of Clauses 1-19, wherein the test and reference subject medical data sets comprise images selected from the group consisting of: a magnetic resonance (MR) image, a computed tomography (CT) image, a single photon emission computed tomography (SPECT) image, a positron emission tomography (PET) image, and a microscopy image.
Clause 21: The method of any of Clauses 1-20, further comprising removing at least one extraneous property in the test and reference subject medical data sets.
Clause 22: The method of any of Clauses 1-21, wherein the electronic neural network comprises at least one layer that performs a regression operation to generate the medical concordance score.
Clause 23: The method of any of Clauses 1-22, wherein the electronic neural network has been trained using a multiple instance learning (MIL) model.
Clause 24: A system for generating a medical concordance score from a test subject medical data set using an electronic neural network, the system comprising: a processor; and a memory communicatively coupled to the processor, the memory storing instructions which, when executed on the processor, perform operations comprising: passing the test subject medical data set through an electronic neural network, wherein the electronic neural network has been trained on a set of training data that comprises a plurality of reference subject medical data sets that are each labeled with a medical determination and are each assigned a ground truth concordance score generated by a plurality of experts, wherein a value of a given ground truth concordance score comprises a fraction of the plurality of experts, if any, that are in accord with the medical determination label of a given reference subject medical data set in the plurality of reference subject medical data sets; and outputting from the electronic neural network a concordance score of the medical determination being indicated by the test subject medical data set.
Clause 25: The system of Clause 24, wherein the plurality of experts comprises human experts, machine experts, or a combination of human and machine experts.
Clause 26: The system of Clause 24 or Clause 25, wherein the plurality of experts comprises at least about three, at least about four, at least about five, at least about six, at least about seven, at least about eight, at least about nine, at least about ten, or more experts.
Clause 27: The system of any of Clauses 24-26, wherein the test subject medical data set is labeled with the medical determination prior to or when passing the test subject medical data set through the electronic neural network.
Clause 28: The system of any of Clauses 24-27, wherein the medical determination comprises a diagnosis of a disease, condition, or disorder.
Clause 29: The system of any of Clauses 24-28, wherein the medical determination comprises a prognosis of a disease, condition, or disorder.
Clause 30: The system of any of Clauses 24-29, wherein the medical determination comprises a recommended treatment plan for a diagnosed disease, condition, or disorder.
Clause 31: The system of any of Clauses 24-30, wherein the medical determination comprises an atypical status, a benign status, or a malignant status.
Clause 32: The system of any of Clauses 24-31, wherein the medical determination comprises an in situ melanoma status or an invasive melanoma status.
Clause 33: The system of any of Clauses 24-32, wherein the medical determination comprises a Gleason score.
Clause 34: The system of any of Clauses 24-33, wherein the medical determination comprises a survival quantification.
Clause 35: The system of any of Clauses 24-34, wherein the medical determination comprises a therapy response.
Clause 36: The system of any of Clauses 24-35, wherein the medical determination comprises a dermatopathological class, prognosis, or diagnosis.
Clause 37: The system of any of Clauses 24-36, wherein the system orders one or more medical tests for, and/or recommends administering one or more therapies to, the test subject when the concordance score of the medical determination being indicated by the test subject medical data set varies from a predetermined threshold value.
Clause 38: The system of any of Clauses 24-37, wherein the one or more medical tests comprise at least one histological stain of a sample obtained from the test subject.
Clause 39: The system of any of Clauses 24-38, wherein the system recommends discontinuing administering one or more therapies to the test subject when the concordance score of the medical determination being indicated by the test subject medical data set varies from a predetermined threshold value.
Clause 40: The system of any of Clauses 24-39, wherein the system generates or updates at least a portion of a medical report for the test subject when the concordance score of the medical determination being indicated by the test subject medical data set varies from a predetermined threshold value.
Clause 41: The system of any of Clauses 24-40, wherein the test and reference subject medical data sets comprise images of histopathology slides.
Clause 42: The system of any of Clauses 24-41, wherein the images comprise whole slide images (WSI).
Clause 43: The system of any of Clauses 24-42, wherein the test and reference subject medical data sets comprise images selected from the group consisting of: a magnetic resonance (MR) image, a computed tomography (CT) image, a single photon emission computed tomography (SPECT) image, a positron emission tomography (PET) image, and a microscopy image.
Clause 44: The system of any of Clauses 24-43, wherein the system removes at least one extraneous property in the test and reference subject medical data sets.
Clause 45: The system of any of Clauses 24-44, wherein the electronic neural network comprises at least one layer that performs a regression operation to generate the medical concordance score.
Clause 46: The system of any of Clauses 24-45, wherein the electronic neural network has been trained using a multiple instance learning (MIL) model.

While the invention has been described with reference to the exemplary embodiments thereof, those skilled in the art will be able to make various modifications to the described embodiments without departing from the true spirit and scope. The terms and descriptions used herein are set forth by way of illustration only and are not meant as limitations. In particular, although the method has been described by examples, the steps of the method can be performed in a different order than illustrated or simultaneously. Those skilled in the art will recognize that these and other variations are possible within the spirit and scope as defined in the following claims and their equivalents. All patents, patent applications, other publications or documents, and the like cited herein are incorporated by reference in their entirety for all purposes to the same extent as if each individual item were specifically and individually indicated to be so incorporated by reference.

1. A computer-implemented method of generating a medical concordance score from a test subject medical data set, the method comprising:
passing the test subject medical data set through an electronic neural network, wherein the electronic neural network has been trained on a set of training data that comprises a plurality of reference subject medical data sets that are each labeled with a medical determination and are each assigned a ground truth concordance score generated by a plurality of experts, wherein a value of a given ground truth concordance score comprises a fraction of the plurality of experts, if any, that are in accord with the medical determination label of a given reference subject medical data set in the plurality of reference subject medical data sets; and
outputting from the electronic neural network a concordance score of the medical determination being indicated by the test subject medical data, thereby generating the medical concordance score from the test subject medical data set.

2. The method of claim 1, wherein the plurality of experts comprises human experts, machine experts, or a combination of human and machine experts.

3. (canceled)

4. The method of claim 1, comprising labeling the test subject medical data set with the medical determination prior to or when passing the test subject medical data set through the electronic neural network.

5. The method of claim 1, wherein the medical determination comprises a diagnosis of a disease, condition, or disorder.

6. The method of claim 1, wherein the medical determination comprises a prognosis of a disease, condition, or disorder.

7. The method of claim 1, wherein the medical determination comprises a recommended treatment plan for a diagnosed disease, condition, or disorder.

8. (canceled)

9. (canceled)

10. (canceled)

11. The method of claim 1, wherein the medical determination comprises a survival quantification.

12. The method of claim 1, wherein the medical determination comprises a therapy response.

13. (canceled)

14. The method of claim 1, further comprising ordering one or more medical tests for, and/or administering one or more therapies to, the test subject when the concordance score of the medical determination being indicated by the test subject medical data set varies from a predetermined threshold value.

15. The method of claim 14, wherein the one or more medical tests comprise at least one histological stain of a sample obtained from the test subject.

16. The method of claim 1, further comprising discontinuing administering one or more therapies to the test subject when the concordance score of the medical determination being indicated by the test subject medical data set varies from a predetermined threshold value.

17. The method of claim 1, further comprising generating or updating at least a portion of a medical report for the test subject when the concordance score of the medical determination being indicated by the test subject medical data set varies from a predetermined threshold value.

18. (canceled)

19. (canceled)

20. (canceled)

21. (canceled)

22. (canceled)

23. (canceled)

24. A system for generating a medical concordance score from a test subject medical data set using an electronic neural network, the system comprising:
a processor; and
a memory communicatively coupled to the processor, the memory storing instructions which, when executed on the processor, perform operations comprising:
passing the test subject medical data set through an electronic neural network, wherein the electronic neural network has been trained on a set of training data that comprises a plurality of reference subject medical data sets that are each labeled with a medical determination and are each assigned a ground truth concordance score generated by a plurality of experts, wherein a value of a given ground truth concordance score comprises a fraction of the plurality of experts, if any, that are in accord with the medical determination label of a given reference subject medical data set in the plurality of reference subject medical data sets; and
outputting from the electronic neural network a concordance score of the medical determination being indicated by the test subject medical data set.

25. The system of claim 24, wherein the plurality of experts comprises human experts, machine experts, or a combination of human and machine experts.

26. (canceled)

27. (canceled)

28. (canceled)

29. (canceled)

30. The system of claim 24, wherein the medical determination comprises a recommended treatment plan for a diagnosed disease, condition, or disorder.

31. (canceled)

32. (canceled)

33. (canceled)

34. (canceled)

35. (canceled)

36. (canceled)

37. The system of claim 24, wherein the system orders one or more medical tests for, and/or recommends administering one or more therapies to, the test subject when the concordance score of the medical determination being indicated by the test subject medical data set varies from a predetermined threshold value.

38. (canceled)

39. The system of claim 24, wherein the system recommends discontinuing administering one or more therapies to the test subject when the concordance score of the medical determination being indicated by the test subject medical data set varies from a predetermined threshold value.

40. The system of claim 24, wherein the system generates or updates at least a portion of a medical report for the test subject when the concordance score of the medical determination being indicated by the test subject medical data set varies from a predetermined threshold value.

41. The system of claim 24, wherein the test and reference subject medical data sets comprise images of histopathology slides.

42. (canceled)

43. The system of claim 24, wherein the test and reference subject medical data sets comprise images selected from the group consisting of: a magnetic resonance (MR) image, a computed tomography (CT) image, a single photon emission computed tomography (SPECT) image, a positron emission tomography (PET) image, and a microscopy image.

44. (canceled)

45. (canceled)

46. (canceled)
